(19) United States
(12) Patent Application Publication
(10) Pub. No.: US 2026/0248532 A1
(43) Pub. Date: Aug. 27, 2026

(54) Adjustable Awl

(71) Applicant: K2M, Inc., Leesburg, VA (US)

(72) Inventor: Liam Patrick Barnes, Round Hill, VA (US)

(73) Assignee: K2M, Inc., Leesburg, VA (US)

(21) Appl. No.: 19/552,537

(22) Filed: Feb. 27, 2026

Related U.S. Application Data
(60) Provisional application No. 63/764,181, filed on Feb. 27, 2025.

(51) Int. Cl. A61B 17/34; A61B 90/00; A61B 17/00

(52) U.S. Cl. CPC A61B 17/3472; A61B 90/08; A61B 2017/00367; A61B 2090/0807

(57) ABSTRACT

An awl including a shaft, a housing at least partially encompassing the shaft, a spring within the housing, and a slider coupled with the shaft and the housing. The shaft having a distal end, a proximal end, and a bone engaging tip at the distal end. The housing having a distal end, a proximal end, and a plurality of first engagement members adjacent to the proximal end, wherein the shaft is slidable within the housing. The spring is configured to bias the shaft with respect to the housing. The slider includes a second engagement member in engagement with at least one of the first engagement members. The movement of the slider with respect to the housing causes the second engagement member to engage another of the first engagement members and movement of the shaft with respect to the housing.

### CROSS-REFERENCE TO RELATED APPLICATIONS

This application claims the benefit of the filing date of United States Provisional Patent Application No. 63/764,181 filed Feb. 27, 2025, the disclosure of which is hereby incorporated herein by reference in its entirety.

### BACKGROUND OF THE INVENTION

Orthopedic surgery often involves the placement of screws or other fastening members in bone. For instance, fracture fixation plates and certain spinal implants are affixed to bone through the use of bone screws. This, however, requires the preparation of the bone before inserting the fastening member.

For instance, holes are often required to be created or enlarged to receive a bone fastener such as a screw. It is desirable for such holes to be created or enlarged precisely and accurately to avoid unwanted complications as well as to promote the intended results of the inserted medical device. Such holes can be created or enlarged with, for instance, an awl.

However, awls are not without their potential drawbacks. When creating or enlarging holes of a bone, slippage can occur in which the holes are of an incorrect position or an incorrect depth. An inaccurate hole can cause for a medical device to be uninsertable (e.g., not enough room for another hole, bone instability, etc.) as well as cause unwanted contact with other biological matter (e.g., spinal cord, nerves, intramedullary canal, etc.).

Therefore, there exists a need for an improved awl, particularly for use in the spine.

### BRIEF SUMMARY OF THE INVENTION

The present disclosure describes examples of awls which are configured to create or enlarge holes in bone, including vertebrae of a spine. The awls include a shaft, a spring assembly, a housing with the shaft and spring assembly configured to be inserted therein, and a slider assembly. The slider assembly is configured to translate along a plurality of engagement members of the housing such that the length at which the shaft can protrude from the housing is limited. Such design is configured to mitigate the possibility of slippage of the shaft depth, thereby increasing accuracy and precision and reducing the chance of unwanted contact with unintended biological matter.

The various disclosed embodiments include a system and method for an awl and its use in a spine surgery. The awl includes a housing, a shaft within the housing configured to contact a bone, a spring assembly within the housing, and a slider assembly coupled with the housing. A plurality of first engagement members of the housing are configured to contact a second engagement member of the slider assembly such that the shaft has a maximum length it can protrude from the housing. The maximum length the shaft can protrude from the housing depends on which groove of the plurality of first engagement members the slider assembly is positioned on. Although discussed herein in connection with use in the spine, awls according to the present disclosure could be utilized in other orthopedic surgeries throughout the body.

In accordance with an aspect of the present disclosure, an awl includes a shaft, a housing at least partially encompassing the shaft, a spring within the housing, and a slider coupled with the shaft and the housing. The shaft has a distal end, a proximal end, and a bone engaging tip at the distal end. The housing has a distal end, a proximal end, and a plurality of first engagement members adjacent to proximal end. The shaft being slidable within the housing. The spring biasing the shaft with respect to the housing. The slider including a second engagement member in engagement with at least one of the first engagement members. The movement of the slider with respect to the housing causes the second engagement member to engage another of the first engagement members and movement of the shaft with respect to the housing.

In other embodiments of this aspect, the awl may include a spring within the housing, the spring biasing the shaft with respect to the housing. The slider of the awl may include an actuator. The actuator may cause the second engagement member to disengage with the first engagement member when an actuation is applied. The awl may further include a longitudinal axis extending at least between the distal end and the proximal end of the shaft. Disengagement of the engagement members may cause the slider to be translatable along the longitudinal axis. The shaft may further include a knob adjacent to the proximal end, the knob having a distal-facing side configured to contact a proximal-facing side of the slider. Contact between the distal-facing side and the proximal-facing side may cause the shaft to be translatively constrained in a distal direction along the longitudinal axis. The awl may further include a collar along a portion of the shaft having a larger cross-section than a majority of the shaft and a pin placed within the housing transverse to the shaft such that the shaft is translatively constrained in a proximal direction when the collar contacts the pin. The housing of the awl may include an oblong aperture along a length of a surface of the housing. The slider may include an oblong aperture along a length of a surface of the slider. The awl may further include a securement member, the securement member detachably coupled with the housing and is configured to secure the spring within the housing. The awl may further include indicia indicating a maximum protrudable length of the shaft.

In accordance with another aspect of the present disclosure, a method for a surgical procedure utilizing an awl includes: actuating an actuator assembly to permit a slider of an awl to translate along a housing; translating the slider to a first position; locking the slider in the first position to set a protrusion depth of a portion of a shaft; and impacting the awl to dispose the portion of a shaft in bone to the protrusion depth.

In other embodiments of this aspect, the locking step further includes engaging a slider engagement member with one of a plurality of housing engagement members of an awl housing. The shaft protrusion depth may be a maximum depth the portion of the shaft can protrude from the awl. The translating step may further include translating the slider to a position indicated by indicia. The method of utilizing the awl may further include the steps of: identifying an orthopedic injury to a spine suitable for operation; and determining vertebrae to be contacted by the awl. The method may further include the steps of: re-actuating the actuator assembly; translating the slider to a second position along the housing; and locking the slider in the second position. The actuation step may further include depressing a button. The shaft may be spring biased.

In accordance with another aspect of the present disclosure, an awl housing includes a housing configured to at least partially encompass a shaft. The housing having a distal end, a proximal end, a plurality of first engagement members adjacent to the proximal end, an axial bore extending from the proximal end to the distal end, and a cavity along a portion of the axial bore configured to house a spring. The plurality of first engagement members are configured to be in engagement with a second engagement member of a slider configured to be coupled with the housing and the shaft. The engagement between one of the first engagement members and the second engagement member causes the shaft to be protrudable from the housing a maximum of a first length. The engagement between another of the first engagement members with the second engagement member causes the shaft to be protrudable from the housing a maximum of a second length, the second length being different than the first length.

In other embodiments of this aspect, the first plurality of engagement members may include a plurality of ridges and a plurality of grooves, each groove being positioned between two ridges, the plurality of grooves configured to receive a ridge of the second engagement member. The plurality of grooves may include five grooves. The housing may further include oblong apertures along a surface of the housing configured to receive cleaning and sterilization instruments. The housing may further include a pin bore configured to house a pin. The pin configured to impede the shaft to move in a first direction, the pine bore being transverse to the axial bore. The housing may further include a first cross-section and a second cross-section extending along a majority of a length of the housing, the first cross-section being closer to the distal end, the second cross-section being closer to the proximal end, wherein a diameter of the first cross-section is lesser than a diameter of the second cross-section. The housing may include a length, wherein a majority of the length of the housing is cylindrical.

### BRIEF DESCRIPTION OF THE DRAWINGS

A more complete appreciation of the subject matter of the present invention and of the various advantages thereof can be realized by reference to the following detailed description in which reference is made to the accompanying drawings in which:

FIG. 1 is an exploded view of an awl according to an embodiment of the present disclosure.

FIG. 2A is a perspective view of a top portion of the awl of FIG. 1.

FIG. 2B is a perspective view of a bottom portion of the awl of FIG. 1.

FIG. 3A is a cross-sectional view of the awl of FIG. 1 in a first position.

FIG. 3B is a cross-sectional view of the awl of FIG. 1 in a second position.

FIG. 4 is an enlarged view of the proximal end of the awl of FIG. 1.

FIG. 5 is an exploded view of an awl according to another embodiment of the present disclosure.

FIG. 6 is a perspective view of an awl according to another embodiment of the present disclosure.

FIG. 7 is another perspective view of the awl of FIG. 6.

FIG. 8 is a cross-sectional view of the awl of FIG. 6.

FIG. 9 is a flowchart illustrating a method for performing a surgical operation utilizing an awl according to the present disclosure.

FIG. 10 is another flowchart illustrating a method for utilizing an awl according to the present disclosure.

### DETAILED DESCRIPTION

In describing the preferred embodiments of the inventions, specific terminology will be used for the sake of clarity. However, the inventions are not intended to be limited to any specific terms used herein, and it is to be understood that each specific term includes all technical equivalents, which operate in a similar manner to accomplish a similar purpose. In the drawings and in the description which follows, the term “proximal” refers to the end of instrument, or a portion thereof, which is closest to the operator in use, while the term “distal” refers to the end of the instrument, or portion thereof, which is farthest from the operator in use. When referring to the human body, the term “proximal” means closer to the heart, the term “distal” means more distant form the heart, the term “anterior” means towards the front part of the body or the face and the term “posterior” means towards the back of the body. The term “medial” means toward the midline of the body and the term “lateral” means away from the midline of the body.

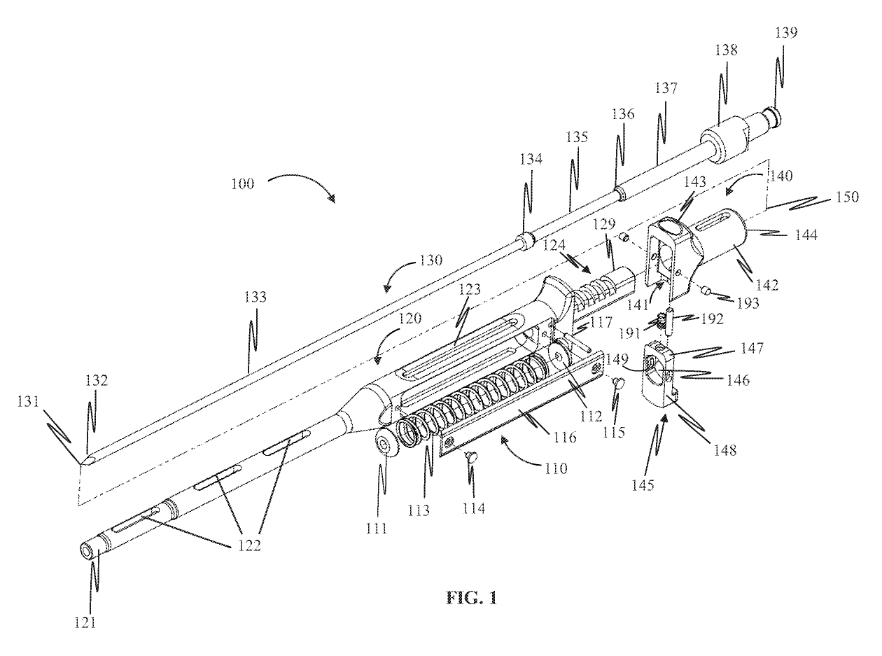

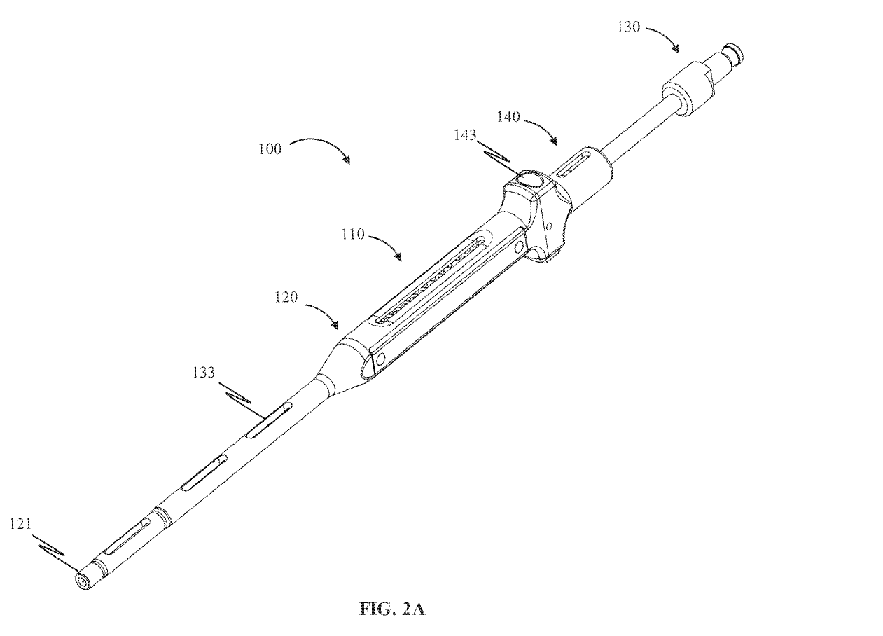

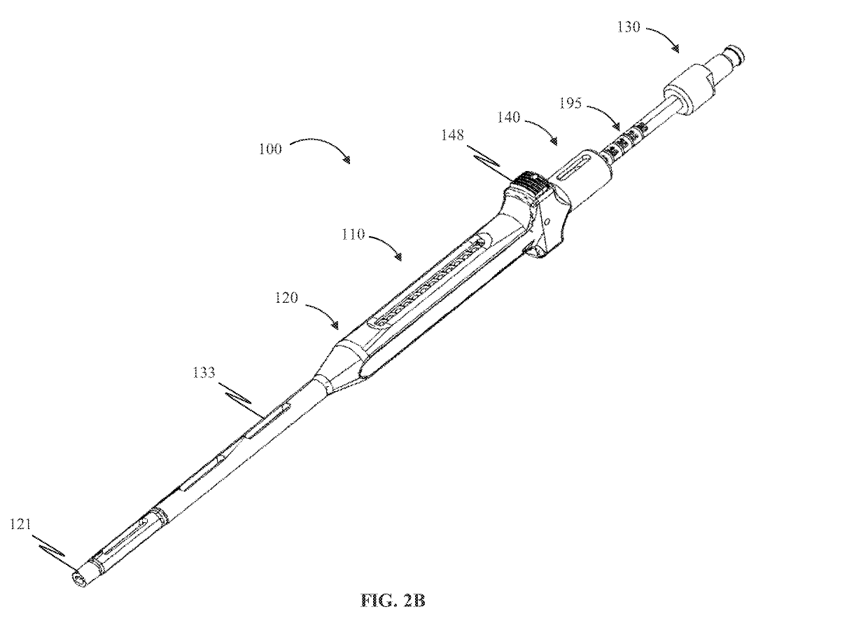

FIG. 1 is an exploded view of an awl 100 according to one embodiment of the present disclosure. The awl depicted includes a spring assembly 110, a housing 120, a shaft 130, and a slider assembly 140. Each of these main components of awl 100 include their own features/components, which will be discussed more fully below.

Housing 120 includes a distal end 121, a proximal end 129, and a plurality of first engagement members 124 adjacent to proximal end 129. Distal end 121 and proximal end 129 extend from a length of housing 120 along a longitudinal axis 150. As shown, housing 120 is substantially cylindrical with two different cross-sections along a majority of its length, wherein one cross-section closer to distal end 121 is lesser than another cross-section closer to proximal end 129. The purpose for the cross-section closer to distal end 121 being narrower than the cross-section closer to the proximal end 129 is at least for the reason that it allows for greater ease of access to the surgical space and better visualization of the surgical space.

Housing 120 further includes a plurality of distal slots 122 and a proximal slot 123. In an embodiment, distal slots 122 and proximal slot 123 are configured to receive cleaning and sterilization instruments such that awl 100 can be cleaned and sterilized.

Housing 120 is configured to at least partially encompass shaft 130, which is configured to contact biological matter and create or enlarge holes therein. Contacted biological matter can include, but is not limited to, bone, skin, fat, muscle, connective tissue. For instance, shaft 130 as shown is designed to create holes in vertebrae. Shaft 130 includes a distal end 131, a bone engaging tip 132 adjacent to distal end 131, a collar portion 134, a grooved edge 136, a proximal end 139, and a knob 138 adjacent to proximal end 139. Bone engaging tip 132 is configured to make contact with a portion of the vertebra (e.g., the pedicle), and cause pilot holes to be created or enlarged.

Shaft 130 is configured to be slidable within housing 120, wherein bone engaging tip 131 is configured to protrude out of and retract into distal end 121 of housing 120. Particularly, awl 100 is configured to have a force applied such that shaft 130 can move in proximal-to-distal and distal-to-proximal directions. According to an embodiment, the force is applied at or near proximal end 139. Proximal end 139 is configured to be engaged by an impactor, but can also be engaged by a human hand, a mechanical hand, or any other tool known in the art. In one embodiment, proximal end 139 is configured to receive a handle attachment in which the handle attachment is impacted causing shaft 130 to move in a proximal-to-distal direction. Moreover, shaft 130 is configured to be rotatable about longitudinal axis 150. Rotation of shaft 130 is advantageous at least because it allows for easier creation or enlargement of holes as well as easier removal of shaft 120 from holes that have been punched.

Shaft 130 also includes a first portion 133 extending between distal end 131 and collar portion 134, a second portion 135 extending between collar portion 134 and grooved edge 136, and a third portion 137 extending between grooved edge 136 and knob 138. All three of first portion 133, second portion 135, and third portion 137 are cylindrical wherein first portion 133 has the smallest diameter, second portion 135 has a larger diameter than first portion 133, and third portion 137 has a larger diameter than both first portion 133 and second portion 135. Third portion 137 includes a smaller diameter than knob 138. As will be discussed more fully below, the different diameters dictate the manner in which shaft 130 interacts with other components of awl 100.

Slider assembly 140 is coupled with housing 120 and shaft 130 and is configured to be coaxial with housing 120 about longitudinal axis 150. Slider assembly 140 includes a body 142, a distal end 141, a proximal end 144, a surface 143 adjacent to distal end 141, an actuator assembly 145, and a slot 194. Slider assembly 140 is configured to house actuator assembly 145 adjacent to distal end 141 wherein actuation of actuator assembly 145 is configured to engage/disengage with first engagement members 124 such that slider assembly 140 can translate along longitudinal axis 150 about housing 120. Slot 194 is configured to receive cleaning and sterilization instruments similar to slots 122-123.

Actuator assembly 145 is coupled with slider assembly 140 via a spring 191, a vertical pin 192, and horizontal pins 193. Vertical pin 192 is configured to stabilize actuator assembly 145 with respect to slider assembly 140 such that actuator assembly 145 is substantially vertical including during translation and actuation. Horizontal pins 193 are configured to stabilize actuator assembly 145 with respect to slider assembly 140 such that a plane of actuator assembly 145 is substantially perpendicular with longitudinal axis 150.

Actuator assembly 145 includes an upper portion 147, a lower portion 148, an aperture 146 formed through the assembly between upper portion 147 and lower portion 148, and a second engagement member 149 being at least a surface between aperture 146 and upper portion 147. Aperture 146 is configured to be coaxial with distal end 141 in the fully assembled position. Lower portion 148 is configured to receive a force thereon, which causes actuator assembly 145 to move within body 142. Particularly, lower portion 148 includes a grooved face configured to receive a human finger. The grooved surface is configured to prevent a finger from slipping. Spring 191 is positioned between upper portion 147 and an inner upper surface of slider assembly 140 such that actuation of actuator assembly 145 causes compression of spring 191. Compression of spring 191 causes actuator assembly 145 to translate upwards (i.e., towards surface 143) such that second engagement member 149 disengages from first engagement member 124 in which slider assembly 140 can translate along longitudinal axis 150 about housing 120. In an embodiment, surface 143 is configured to be contacted during the application of force on actuator assembly 145 such as for, but not limited to, leverage. As a non-limiting example, an operator’s thumb may contact lower portion 148 whereas the operator’s other fingers (e.g., index finger) of the same hand contacts grip 143 such that the operator’s hand is in a pinching-like grip.

Second engagement member 149 of actuator assembly 145 is configured to engage with first engagement members 124. The engagement members comprise gear-like or toothed protrusions and depressions such that a protrusion of second engagement member 149 can be engaged with a depression of first engagement members 124 and vice versa.

Slider assembly 140 is configured to translate about longitudinal axis 150. Specifically, slider assembly 140 is configured to translate between different grooves of first engagement members 124. Slider assembly 140 can move between first engagement members 124 by a force to lower portion 148, if engaged, to disengage the first and second engagement members followed by a translative force in at least a direction parallel to longitudinal axis 150. If slider assembly 140 is in a position between different first engagement members 124, a force to lower portion 148 is not required and slider assembly 140 can translate to an adjacent first engagement member 124.

The force applied to translate slider assembly 140 can be, but is not limited to, body 142 by a surgeon or other medical professional. As a non-limiting example, an index finger (or any other non-thumb finger) of a first hand contacts button 143 while a thumb of the first hand contacts the grooved face of lower portion 148 in a pinching-like grip. As another non-limiting example, the palm and fingers of a hand may wrap around body 142 such that the thumb of the same hand may also press surface 143 transverse to longitudinal axis 150. The translation of slider assembly 140 along longitudinal axis 150 may be in either proximal-to-distal or distal-to-proximal directions.

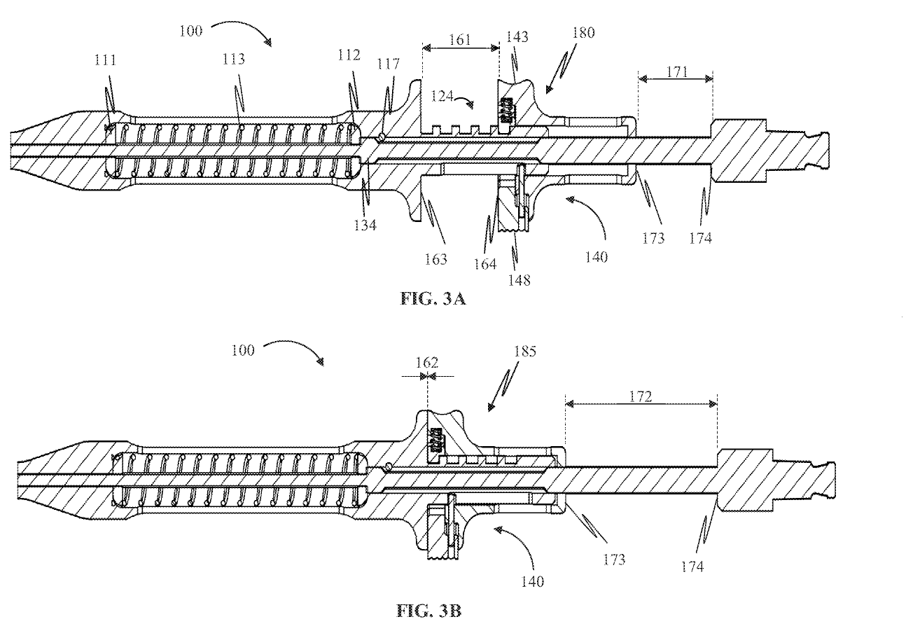

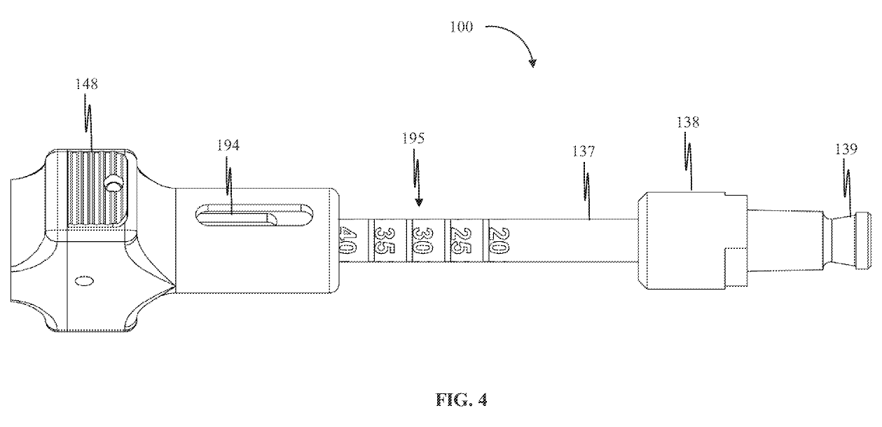

Translation of slider assembly 140 along first engagement members 124 adjusts the total length that shaft 130 can protrude from distal end 121. As shown in connection with FIGS. 3A-B, knob 138 includes a distal face 174 and slider assembly 140 includes a proximal face 173 adjacent to proximal end 144. Proximal face 173 of slider assembly 140 is configured to impede shaft 130 from translating a certain amount in a proximal-to-distal direction. Particularly, as shaft 130 moves distally, distal face 174 of knob 138 is configured to contact proximal face 173 of slider assembly 140. Depending on the positioning of slider assembly 140 on the first engagement members 124, knob 138 can go further (or shorter) along longitudinal axis 150 before contacting the proximal face of slider assembly 140. As a non-limiting example, FIGS. 3A-3B show two different positions second engagement member can be engaged with first engagement members 124 (first position 180 and second position 185). This is advantageous at least because it prevents an inadvertent increase in the depth that shaft 130 can extend into the bone. Put another way, an operator of awl 100 that sets slider assembly 140 at a certain groove of first engagement members 124 knows that shaft 130 cannot protrude out of distal end 121 beyond a predetermined length. The protrudable length of shaft 130 from distal end 121 can be conveyed to the user by indicia coupled with awl 100 indicating a maximum protrudable length of shaft 130. In one embodiment, the indicia are markings 195 on third portion 137 of shaft 130, as shown by FIG. 4. Markings 195 include numerals and lines, which indicate the maximum protrudable length of shaft 130 and the lines correspond to those numerals indicating where exactly each length is. The line adjacent to proximal end 144 of slider assembly 140 indicates the maximum protrudable length of shaft 130 when shaft 130 is in a fully retracted position. In an embodiment, the protrudable length can also be determined by visually inspecting which first engagement member 124 second engagement member 149 is engaged with or measuring the maximum length (e.g., 171, 172) between proximal face 173 and distal face 174.

The translation of shaft 130 is at least partially acted upon by spring assembly 110. Spring assembly 110 includes a distal end cap 111, a proximal end cap 112, a compression member 113, a first fastener 114, a second fastener 115, a securement member 116, and a pin 117. Distal end cap 111, proximal end cap 112, and compression member 113 are configured to be located within housing 120 and coaxial with housing 120 about longitudinal axis 150. Both distal end cap 111 and proximal end cap 112 have a substantially semicircular toroid shape and are positioned at opposite ends of compression member 113 along longitudinal axis 150. Each of distal end cap 111 and proximal end cap 112 have a larger flat face facing towards compression member 113 and a smaller flat face facing away from compression member 113. The larger flat faces of end caps 111-112 are configured to contact a portion of compression member 113 such that a force applied to end caps 111-112 are configured to translate to compression member 113 and vice versa. As a non-limiting example, a force applied in a proximal-to-distal direction along longitudinal axis 150 would cause proximal end cap 112 to translate the force to compression member 113 and subsequently to distal end cap 111 in which compression member 113 would compress (assuming distal end cap 111 could not move further distally). The smaller flat face of proximal end cap 112 is configured to contact collar portion 134 of shaft 130 such that as shaft 130 moves in a proximal-to-distal direction, collar portion 134 can contact the smaller flat face of proximal end cap 112 causing compression member 113 to compress. This is desirable at least because a force required to move bone engaging tip 132 distally would need to be greater than the spring force of compression member 113. Moreover, the further bone engaging tip 132 protrudes distally, the greater the force required to move shaft 130 becomes (i.e., Hooke’s Law) which is desirable as the further bone engaging tip 132 protrudes distally, the greater the risk of unwanted contact (e.g., spinal cord, etc.).

Securement member 116 is configured to be attached to housing 120 with first fastener 114 and second fastener 115. When end caps 111-112 and compression member 113 are inserted within housing 120, securement member 116 is configured to secure end caps 111-112 and compression member 113 therein. In an embodiment, securement member 116 is also configured to be detached from housing 120. The purpose for securement member 116 is at least for assembly purposes such that components of awl 100 (e.g., 111-115) can be placed within housing 120.

Pin 117 is configured to be placed within a bore of housing 120 wherein pin 117 partially blocks the bore. When pin 117 is inserted into housing 120, it is configured to limit the range of translative motion along longitudinal axis 150 of shaft 130. Particularly, pin 117 is positioned in a portion of the bore configured to house second portion 135 of shaft 130 such that collar portion 134 and raised groove 136 are impeded from crossing over pin 117 in their respective directions. For example, collar portion 134 cannot go beyond pin 117 in a distal-to-proximal direction along longitudinal axis 150 and raised groove 136 cannot go beyond pin 117 in a proximal-to-distal direction along longitudinal axis 150.

FIG. 3A shows a cross-sectional view of awl 100 in a first position 180. As depicted, housing 120 includes a proximal face 163 adjacent to distal end 129 and slider assembly 140 includes a distal face 164 adjacent to distal end 141. A first distance 161 separates proximal face 163 with distal face 164. First distance 161 is the largest distance proximal face 163 and distal face 164 can be separated while slider assembly 140 is positioned along first engagement members 124. As shown, first engagement members 124 include five different grooves which second engagement member 149 of slider assembly 140 is configured to engage with. Thus, there are at least five distances in which proximal face 163 and distal face 164 can be apart while slider assembly 140 is engaged with first engagement members 124. In some embodiments, first engagement members 124 are configured to have more or less grooves in which slider assembly 140 can be engaged with and positioned on. In another embodiment, the engagement between first engagement members 124 and second engagement member 149 are communicatively connected with indicia indicating specific intervals of distance associated with each groove of the first engagement members. Specifically, the indicia are configured to provide a measurement of maximum distance shaft 130 can protrude from distal end 121 of housing 120 for each respective first engagement member 124 second engagement member 149 engages with.

Depending on which groove of first engagement members 124 slider assembly 140 is positioned on determines a second distance between proximal face 173 of slider assembly 140 and distal face 174 of knob 138. As shown, at first distance 161, the maximum distance between proximal face 173 and distal face 174 is second distance 171. This is at least true because pin 117 impedes collar portion 134 of shaft 130 from moving more proximally along the bore of housing 120. Second distance 171 is also the maximum distance which shaft 130 can protrude out from distal end 121 when shaft 130 is pushed distally.

As shown, distal end cap 111 and proximal end cap 112 are positioned within a portion of the bore configured to house compression member 113. As shaft 130 moves in a proximal-to-distal direction, collar portion 134 is configured to contact proximal end cap 112 such that compression member 113 compresses and proximal end cap 112 translates distally. As shaft 130 moves in a distal-to-proximal direction, proximal end cap 112 is configured to contact collar portion 134 and translate shaft 130 proximally. Shaft 130 is configured to translate proximally until collar portion 134 is in contact with pin 117 in which shaft 130 is in its furthest intended retractable position.

FIG. 3B is a cross-sectional view of awl 100 in a second position 185. As shown, slider assembly 140 is engaged with a groove of first engagement members 124 nearest to distal face 163 such that a first distance 162 between faces 163 and 164 is approximately zero. Comparatively, second distance 172 is the largest maximum distance between faces 173 and 174. However, the distance between distal face 174 and proximal face 173 can be any distance between second distance 172 and a distance where faces 173 and 174 are in contact with each other. Second position 185 is also the position in which shaft 130 can protrude the most from distal end 121.

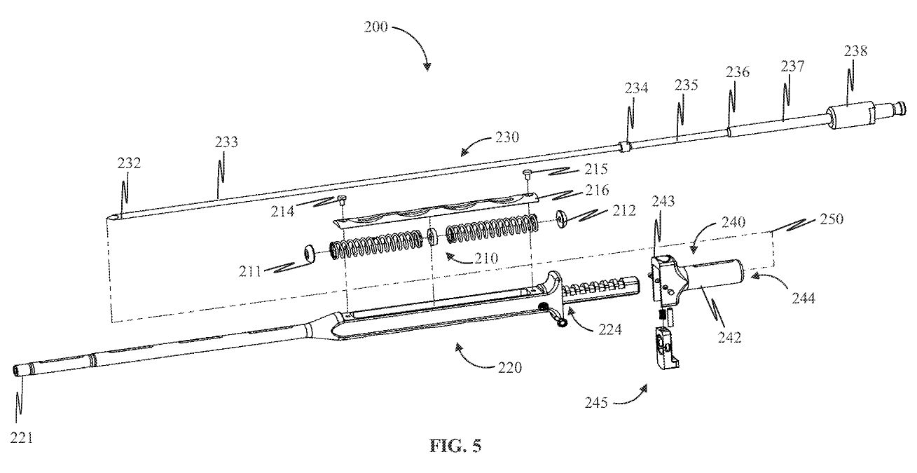

FIG. 5 is an exploded view of an awl 200 according to another embodiment of the present disclosure. Awl 200 is similar to awl 100, and therefore like elements referred to with similar numerals within 200-series of numbers. For example, awl 200 includes a housing 220 coupled with a slider assembly 240 and a shaft 230 in which shaft 230 is configured to translate out of and into a distal end 221 of housing 220. However, unlike awl 100, awl 200 includes an opening on housing 220 for spring assembly 210 to be housed at least partially therein on a top face of housing 220 facing substantially the same direction as surface 243. Thus, awl 200 does not include a proximal slot along a top face of housing 220 like awl 100 (proximal slot 123).

Furthermore, awl 200 includes first engagement members 224 with at least seven different grooves in which a second engagement member of slider assembly 240 is configured to engage with. Depending on which groove of first engagement members 224 slider assembly 240 is engaged with determines the maximum length shaft 230 can protrude from distal end 221 of housing 220.

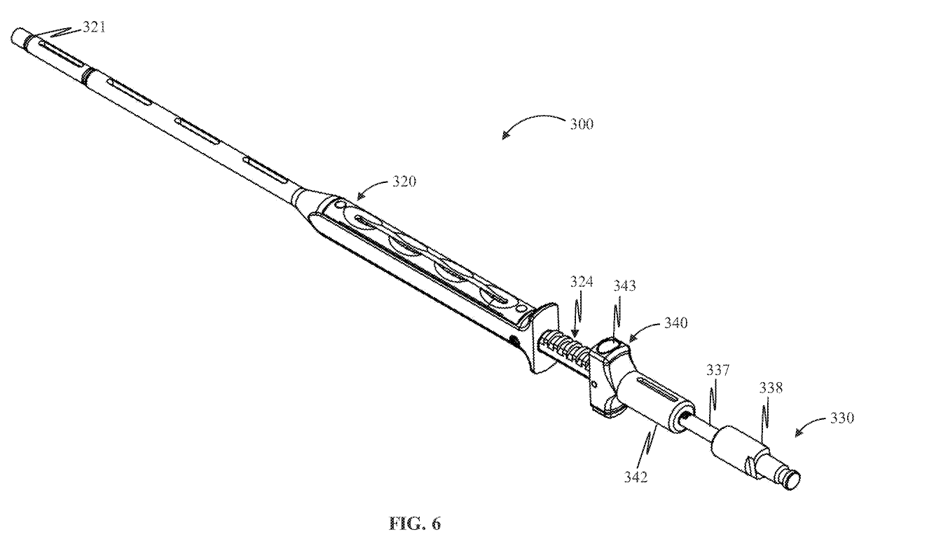

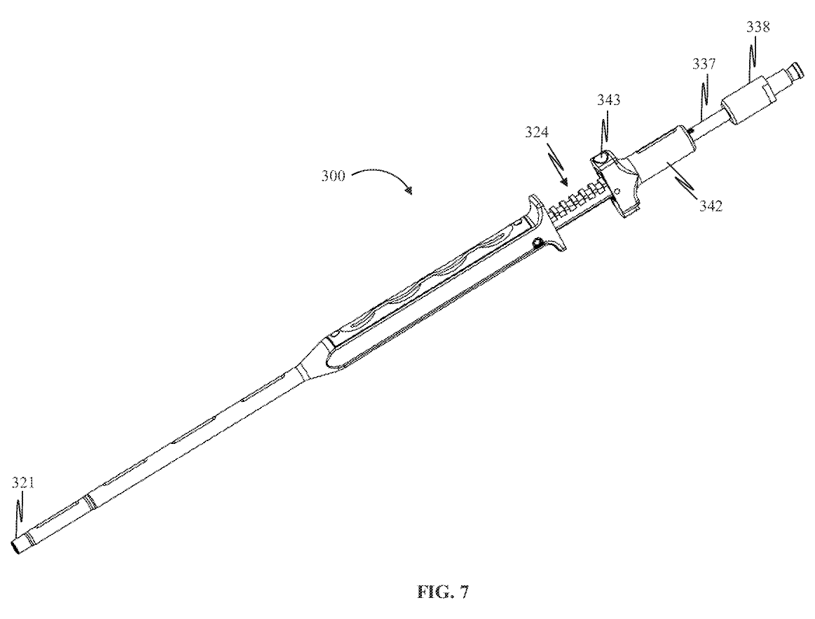

FIG. 6 is a first perspective view of an awl 300 according to another embodiment of the present disclosure. Awl 300 is similar to awl 100 and awl 200, and therefore like elements are referred to with similar numerals within 300-series of numbers. For example, awl 300 includes a housing 320 coupled with a slider assembly 340 and a shaft 330 in which shaft 330 is configured to translate out of and into a distal end 321. However, unlike awl 100, awl 300 does not include an opening on housing 320 configured to receive a securement member therein. As such, spring assembly 310 is configured to be substantially fixed within housing 320.

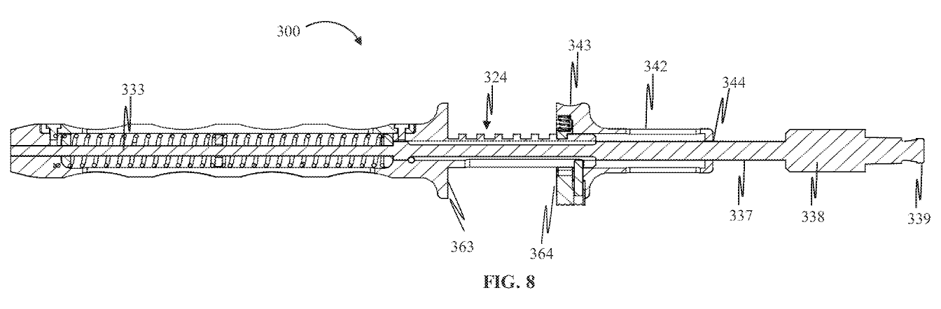

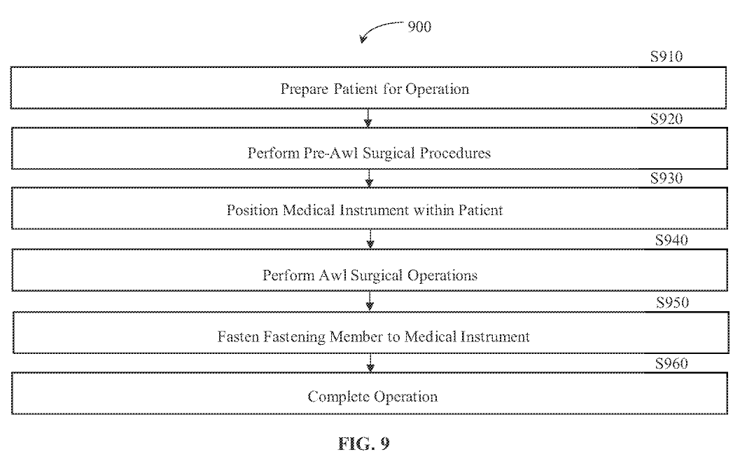

FIG. 8 is a cross-sectional view of awl 300 according to an embodiment. As shown, spring assembly 310 does not include end caps positioned adjacent to compression member 314 within housing 320.

FIG. 9 is a flowchart illustrating a method for performing a surgical operation utilizing an awl according to an embodiment.

At S910, a patient is prepared for an operation such as, but not limited to, spinal fusion wherein a plate is inserted between two vertebral bodies. The preparation includes all procedures known in the art to prepare the patient for surgery. These can include anesthetizing the patient, positioning the patient on the operating table (e.g., in a supine position), sterilizing the patient, performing any other known pre-operation procedures, a combination thereof, and the like.

At S920, pre-awl surgical procedures are performed. Pre-awl surgical procedures can include, but are not limited to, creating an incision on the patient, grafting bone to receive a medical instrument thereon, tapping bone with a threaded screw hole, removing any debris from at least the surgical site, any combination thereof, and the like.

At S930, a medical instrument is positioned within the patient. In an embodiment, the medical instrument is a plate configured to be coupled with two adjacent vertebral bodies.

At S940, awl surgical operations are performed. A bone engaging tip of an awl is configured to make contact with a desired bone of a surgical site. Moreover, after contacting the bone, the awl is configured to create or enlarge holes configured to receive fastening members therein. For instance, in a spinal fusion with a plate inserter of four screw holes, the awl is configured to create or enlarge four holes at a location where the four screw holes of the plate inserter are configured to be placed.

At S950, the fastening members fasten the medical instrument to the bone. The fastening members can include screws, pins, rivets, bolts, any other fasteners known in the art, a combination thereof, and the like. The fastening members are configured to be received by a hole created or enlarged such as at S940. This is at least desirable as a pilot hole helps guide the fastening members into the bone allowing for precise and accurate fastener placement as well as mitigating the risk of bone splintering and fracturing during the insertion of the fastening members.

At S960, the operation is completed. Completing the operation includes any procedures known in the art necessary to finish the operation (e.g., suturing incisions, removing foreign material from surgical site, etc.), any post-operation procedures, a combination thereof, and the like.

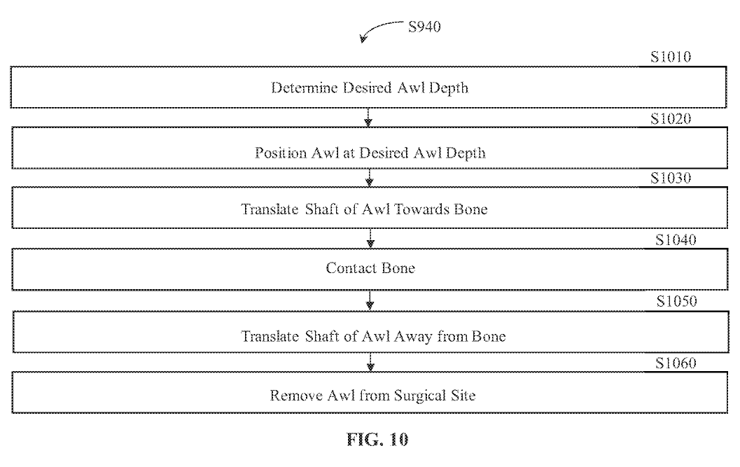

FIG. 10 is a flowchart illustrating a method for utilizing an awl S940 according to an embodiment. In an embodiment, the method may be performed by awl 100, awl 200, or awl 300.

At S1010, a desired awl depth is determined. A desired awl depth may include a depth which allows the shaft of the awl to protrude far enough such that a desired hole depth can be created when the shaft contacts a bone, but not too far as to allow for unwanted slippage of the shaft. An awl depth larger than is required to create or enlarge holes of a bone is undesirable as it exposes the patient to unwanted risks such as slippage of the shaft and uncertainty in the awl operator as to whether they have gone far enough with the awl for the desired hole depth. As a non-limiting example, an awl operator should choose an awl depth of 20 millimeters where an awl has an awl depth of either 10, 20, and 30 millimeters and where the required operation calls for 20 millimeters of shaft protrusion. If for instance, an awl depth of 30 millimeters is selected the operator has to ensure not to go beyond the intended 20 millimeters.

At S1020 the awl is positioned at the desired awl depth. For instance, if it is determined that the awl should be positioned at a third groove of the first engagement member for a procedure, the operator may verify whether the slider assembly of the awl is engaged with the third groove of the first engagement member. The operator can verify whether the slider assembly is engaged with the desired groove by reading indicia communicatively connected with the awl configured to provide maximum shaft depth of the specific groove slider assembly is engaged with. If the slider assembly is engaged with the groove corresponding to the desired awl depth, S1030 may be performed. If the slider assembly is engaged with a groove not corresponding to the desired awl depth, the slider assembly is moved to engage with the groove corresponding to the desired awl depth. The slider assembly is moved by pressing a portion of the slider assembly causing the slider assembly to actuate and disengage with a groove of the first engagement members. When the slider assembly is disengaged with a groove, a translative force can be applied to the slider assembly such that it can become engaged with another groove of the first engagement members, such as the groove of the desired awl depth.

At S1030, the shaft of the awl is translated towards the surgical site. In an embodiment, the operator applies a translative force at the proximal end of the shaft. Accordingly, the distal end of the shaft protrudes from the distal end of the housing. The spring assembly within the housing of the awl is configured to apply a resistive force opposite the translative force such that the deeper the shaft protrudes, the greater the resistive force applied, and thus the greater the force needed to further protrude the shaft. This is desirable as it at least helps mitigate unwanted protrusion of the shaft.

At S1040, the shaft of the awl contacts a desired bone. In an embodiment, the contact with the desired bone creates or enlarges a hole configured to receive a fastening member therein.

At S1050, the shaft of the awl is translated away from the surgical site. In an embodiment, the operator applies a translative force at the proximal end of the shaft to retract the shaft into distal end of the housing. In another embodiment, the compression member of the spring assembly applies a force causing the shaft to retract into the housing.

Depending on whether more holes are required S1030-S1050 can be repeated such that all desired holes can be created. Moreover, S1010-S1020 can also be repeated depending on whether a different awl depth is desired. At S1060, the awl is removed from the surgical site.

It is to be understood that the various components discussed herein can be constructed of any suitable material for use in the human body. For instance, it is contemplated to construct the various components of metallic materials, such as titanium and stainless steel. It is also contemplated to use various manufacturing methods to construct the components, including additive manufacturing.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. An awl comprising:
a shaft having a distal end, a proximal end, and a bone engaging tip at the distal end;
a housing at least partially encompassing the shaft, the housing having a distal end, a proximal end, and a plurality of first engagement members adjacent the proximal end, the shaft being slidable within the housing; and
a slider coupled with the shaft and the housing, the slider including a second engagement member configured to be in engagement with one of the first engagement members, the engagement between one of the first engagement members and the second engagement member causes the shaft to be protrudable from the housing a maximum of a first length,
wherein movement of the slider with respect to the housing causes the second engagement member to engage another of the first engagement members, engagement of another first engagement member causes the shaft to be protrudable from the housing a maximum of a second length.

2. The awl of claim 1, wherein the awl further includes a spring within the housing, the spring biasing the shaft with respect to the housing.

3. The awl of claim 1, wherein the slider includes an actuator, the actuator causing the second engagement member to disengage with the first engagement member when an actuation is applied.

4. The awl of claim 1, the awl further comprising a longitudinal axis extending at least between the distal end and the proximal end of the shaft, wherein disengagement of the engagement members causes the slider to be translatable along the longitudinal axis.

5. The awl of claim 1, wherein the shaft further includes a knob adjacent to the proximal end, the knob having a distal-facing side configured to contact a proximal-facing side of the slider.

6. The awl of claim 5, wherein contact between the distal-facing side and the proximal-facing side causes the shaft to be translatively constrained in a distal direction along the longitudinal axis.

7. The awl of claim 1, wherein the awl further includes a collar along a portion of the shaft having a larger cross-section than a majority of the shaft and a pin placed within the housing transverse to the shaft such that the shaft is translatively constrained in a proximal direction when the collar contacts the pin.

8. The awl of claim 1, wherein the housing includes an oblong aperture along a length of a surface of the housing and the slide includes an oblong aperture along a length of a surface of the slider.

9. The awl of claim 1, wherein the awl further includes a securement member, the securement member detachably coupled with the housing and is configured to secure the spring within the housing.

10. The awl of claim 1, wherein the awl further includes indicia indicating a maximum protrudable length of the shaft.

11. A method for a surgical procedure utilizing an awl, the method comprising:
actuating an actuator assembly to permit a slider of an awl to translate along a housing;
translating the slider to a first position;
locking the slider in the first position to set a protrusion depth of a portion of a shaft; and
impacting the awl to dispose the portion of a shaft in bone to the protrusion depth.

12. The method of claim 11, wherein the locking step includes engaging a slider engagement member with one of a plurality of housing engagement members of an awl housing.

13. The method of claim 11, wherein the shaft protrusion depth is a maximum depth the portion of the shaft can protrude from the awl.

14. The method of claim 11, wherein the translating step includes translating the slider to a position indicated by indicia and the actuating step includes depressing a button.

15. The method of claim 11, the method further comprising:
identifying an orthopedic injury to a spine suitable for operation; and
determining vertebrae to be contacted by the awl.

16. The method of claim 11, further comprising the steps of:
re-actuating the actuator assembly;
translating the slider to a second position along the housing; and
locking the slider in the second position.

17. An awl housing comprising:
a housing configured to at least partially encompass a shaft, the housing having a distal end, a proximal end, a plurality of first engagement members adjacent the proximal end, an axial bore extending from the proximal end to the distal end, and a cavity along a portion of the axial bore configured to house a spring,
wherein the plurality of first engagement members are configured to be in engagement with a second engagement member of a slider configured to be coupled with the housing and the shaft, the engagement between one of the first engagement members and the second engagement member causes the shaft to be protrudable from the housing a maximum of a first length,
wherein engagement between another of the first engagement members with the second engagement member causes the shaft to be protrudable from the housing a maximum of a second length, the second length being different than the first length.

18. The awl housing of claim 17, wherein the first plurality of engagement members include a plurality of ridges and a plurality of grooves, each groove being positioned between two ridges, the plurality of grooves configured to receive a ridge of the second engagement member.

19. The awl housing of claim 17, wherein the housing further includes oblong apertures along a surface of the housing configured to receive cleaning and sterilization instruments and a pin bore configured to house a pin, the pin configured to impede the shaft to move in a first direction, the pin bore being transverse to the axial bore.

20. The awl housing of claim 17, wherein the housing further includes a first cross-section and a second cross-section extending along a majority of a length of the housing, the first cross-section being closer to the distal end, the second cross-section being closer to the proximal end, wherein a diameter of the first cross-section is lesser than a diameter of the second cross-section.
